(19) United States
(12) Patent Application Publication
(10) Pub. No.: US 2017/0127960 A1
(43) Pub. Date: May 11, 2017

(54) METHOD AND APPARATUS FOR ESTIMATING HEART RATE BASED ON MOVEMENT INFORMATION

(71) Applicant: Samsung Electronics Co., Ltd., Suwon-si (KR)

(72) Inventors: Dae-Geun JANG, Yongin-si (KR); Byunghoon KO, Hwaseong-si (KR); SangKon BAE, Seongnam-si (KR)

(73) Assignee: Samsung Electronics Co., Ltd., Suwon-si (KR)

(21) Appl. No.: 15/251,026

(22) Filed: Aug. 30, 2016

(30) Foreign Application Priority Data
Nov. 10, 2015 (KR) 10-2015-0157065

(51) Int. Cl. A61B 5/024; A61B 5/0245; A61B 5/00

(52) U.S. Cl. CPC A61B 5/02438; A61B 5/721; A61B 5/6813; A61B 5/742; A61B 5/0245; A61B 5/02416

(57) ABSTRACT

A method of estimating a heart rate includes: measuring, using a processor, movement information based on a physical activity of a user; and estimating, using the processor, a heart rate of the user by applying the movement information to a preset regression model, wherein the regression model is based on a correlation between heart rates and movement information.

### CROSS-REFERENCE TO RELATED APPLICATIONS

This application claims the benefit under 35 USC 119(a) of Korean Patent Application No. 10-2015-0157065 filed on Nov. 10, 2015, in the Korean Intellectual Property Office, the entire disclosure of which is incorporated herein by reference for all purposes.

### BACKGROUND

1. Field

The following description relates to a method and apparatus for estimating a heart rate based on movement information.

2. Description of Related Art

A heart rate is detected based on an electrocardiogram (ECG) and a pulse wave, which may have various noise sources based on principles of measurement. For example, a baseline wandering is a low-frequency noise component included in the ECG and the pulse wave, and appears in response to, for example, a respiration, a sympathetic nervous system activity, and a thermoregulation. Various signal processing methods have been provided to remove a noise component from the ECG and the pulse wave.

Known signal processing methods may be adapted to regenerate a signal having noise of a relatively short interval, or an unmeasured signal of a relatively short interval. However, when a signal is repetitively unmeasured or noise occurs in the signal in several intervals, a signal processing result may be insufficiently inaccurate and unreliable. Additionally, when a measured biosignal does not include a valid signal such as the ECG and the pulse wave, it may ultimately not be possible to perform a heart rate calculation or analyses with the measured heartrate information.

### SUMMARY

This Summary is provided to introduce a selection of concepts in a simplified form that are further described below in the Detailed Description. This Summary is not intended to identify key features or essential features of the claimed subject matter, nor is it intended to be used as an aid in determining the scope of the claimed subject matter.

According to one general aspect, a method of estimating a heart rate includes: measuring, using a processor, movement information based on a physical activity of a user; and estimating, using the processor, a heart rate of the user by applying the movement information to a preset regression model, wherein the regression model is based on a correlation between heart rates and movement information.

The movement information may include at least one of a measured moving speed or a moving distance for the user.

The regression model may include at least one of a first regression model preset based on a correlation between average heart rates and average moving information of a plurality of users and a second regression model preset based on a correlation between heart rates and at least previous movement information of the user.

The estimating may include estimating the heart rate of the user by applying the movement information to one of the first regression model and the second regression model based on whether a personalized correction is determined to be performed.

The method may further include generating the regression model so as to be personalized for the user based on previous movement information of the user.

The generating may include generating a second regression model by correcting a first regression model based on the previous movement information of the user.

The method may further include measuring the heart rate of the user.

The method may further include compensating for a portion of the heart rate of the user determined to be an unmeasured or mis-measured heart rate section based on the estimated heart rate.

The method may further include evaluating a signal quality of the measured heart rate based on at least one of a difference between the measured heart rate and the estimated heart rate, or a ratio between the measured heart rate and the estimated heart rate.

The method may further include substituting the portion of the measured heart rate with a corresponding portion of the estimated heart rate in response to the signal quality being lower than a preset reference.

The evaluating of the signal quality may further include detecting at least one of an invalid heart rate section or a valid heart rate section from the measured heart rate, based on at least one of the difference and the ratio.

The detecting may include: comparing the difference or the ratio to a preset reference; and determining that a heart rate section of the measured heart rate in which the difference or the ratio has a value greater than the preset reference is the invalid heart rate section, based on a result of the comparing.

The method may further include: analyzing physical activity information of the user based on the estimated heart rate; and providing a feedback on a result of the analyzing.

The method may further include receiving body information of the user, wherein the estimating includes estimating the heart rate of the user by applying the movement information and the body information of the user to the regression model.

The body information may include at least one of a gender, an age, a height, a weight, a body mass index (BMI), or a stable-state heart rate.

A non-transitory computer readable medium may include programmed instructions configured to control one or more processor devices to implement the method.

According to another general aspect, an apparatus for estimating a heart rate incldues: a first measurer configured to measure movement information of a user based on a physical activity of the user; and a processor configured to estimate a heart rate of the user by applying the movement information to a preset regression model, wherein the regression model is based on a correlation between heart rates and movement information.

The apparatus may further include a second measurer configured to measure the heart rate of the user.

The processor may be configured to compensate for the heart rate of the user based on the estimated heart rate, in response to a portion of the heart rate of the user being determined to be unmeasured or measured incorrectly.

The processor may be configured to: evaluate a signal quality of the measured heart rate based on at least one of a difference between the measured heart rate and the estimated heart rate, or a ratio between the measured heart rate and the estimated heart rate; and substitute the portion of measured heart rate with a corresponding portion of the estimated heart rate in response to the signal quality being lower than a preset reference.

Other features and aspects will be apparent from the following detailed description, the drawings, and the claims.

### BRIEF DESCRIPTION OF DRAWINGS

FIGS. 1A illustrates an example of an apparatus for estimating a heart rate.

FIG. 1B illustrates examples of wearable devices or other devices into which the apparatus of FIG. 1A may be incorporated, and a mobile device that may communicate with such wearable devices,

FIGS. 2 through 4 illustrate examples of methods of estimating a heart rate.

FIG. 5 illustrates an example of estimating, storing, analyzing, and outputting a heart rate.

FIG. 6 illustrates an example of a method of estimating a heart rate.

FIGS. 7 through 9 illustrate examples of operations of apparatuses for estimating a heart rate.

FIGS. 10A-10D illustrate graphs representing examples of a correlation between a moving distance and an actual measured heart rate using different regression models through a personalization operation.

FIGS. 11A-11D illustrate graphs representing examples of a correlation between an actual heart rate and an estimated heart rate using different regression models through a generalization operation.

Throughout the drawings and the detailed description, unless otherwise described or provided, the same drawing reference numerals will be understood to refer to the same elements, features, and structures. The drawings may not be to scale, and the relative size, proportions, and depiction of elements in the drawings may be exaggerated for clarity, illustration, and convenience.

### DETAILED DESCRIPTION

The following detailed description is provided to assist the reader in gaining a comprehensive understanding of the methods, apparatuses, and/or systems described herein. However, various changes, modifications, and equivalents of the methods, apparatuses, and/or systems described herein will be apparent to one of ordinary skill in the art. The sequences of operations described herein are merely examples, and are not limited to those set forth herein, but may be changed as will be apparent to one of ordinary skill in the art, with the exception of operations necessarily occurring in a certain order. Also, descriptions of functions and constructions that are well known to one of ordinary skill in the art may be omitted for increased clarity and conciseness.

The features described herein may be embodied in different forms, and are not to be construed as being limited to the examples described herein. Rather, the examples described herein have been provided so that this disclosure will be thorough and complete, and will convey the full scope of the disclosure to one of ordinary skill in the art.

It will be understood that, although the terms first, second, etc. may be used herein to describe various elements, these elements should not be limited by these terms. These terms are only used to distinguish one element from another. For example, a first signal could be termed a second signal, and, similarly, a second signal could be termed a first signal without departing from the teachings of the disclosure.

It will be understood that when an element or layer is referred to as being “on”, “attached to”, or “connected to” another element or layer, it can be directly on or connected to the other element or layer or through intervening elements or layers may be present. In contrast, when an element is referred to as being “directly on”, “directly attached to”, or “directly connected to” another element or layer, there are no intervening elements or layers present. Other words used to describe the relationship between elements or layers should be interpreted in a like fashion (e.g., “between” versus “directly between,” “adjacent” versus “directly adjacent,” “on” versus “directly on”).

The terminology used herein is for the purpose of describing particular examples only and is not to be limiting of the examples. As used herein, the singular forms “a”, “an”, and “the” are intended to include the plural forms as well, unless the context clearly indicates otherwise. It will be further understood that the terms “include/comprise” and/or “have” when used in this specification, specify the presence of stated features, integers, steps, operations, elements, components, and/or combinations thereof, but do not preclude the presence or addition of one or more other features, numbers, steps, operations, elements, components, and/or groups thereof.

Unless otherwise defined, all terms including technical and scientific terms used herein have the same meaning as commonly understood by one of ordinary skill in the art to which examples belong. It will be further understood that terms, such as those defined in commonly-used dictionaries, should be interpreted as having a meaning that is consistent with their meaning in the context of the relevant art and will not be interpreted in an idealized or overly formal sense unless expressly so defined herein.

The following example embodiments may be applied to estimate and compensate for a heart rate of a user by considering a user's activity. Example embodiments may be implemented to be various forms, for example, a personal computer, a laptop computer, a tablet computer, a smartphone, a television, a smart appliance, an intelligent vehicle, and a wearable device. Example embodiments may be applied to provide physical activity information analyzed by estimating a heart rate of a user based on movement information of the user measured using, for example, sensors of a connected or corresponding smartphone, a mobile device, and a smart home system. Example embodiments may also be applied to, for example, a healthcare service for the user where results of the compensated heart rate are displayed or externally provided. Hereinafter, reference will now be made in detail to examples with reference to the accompanying drawings, wherein like reference numerals refer to like elements throughout.

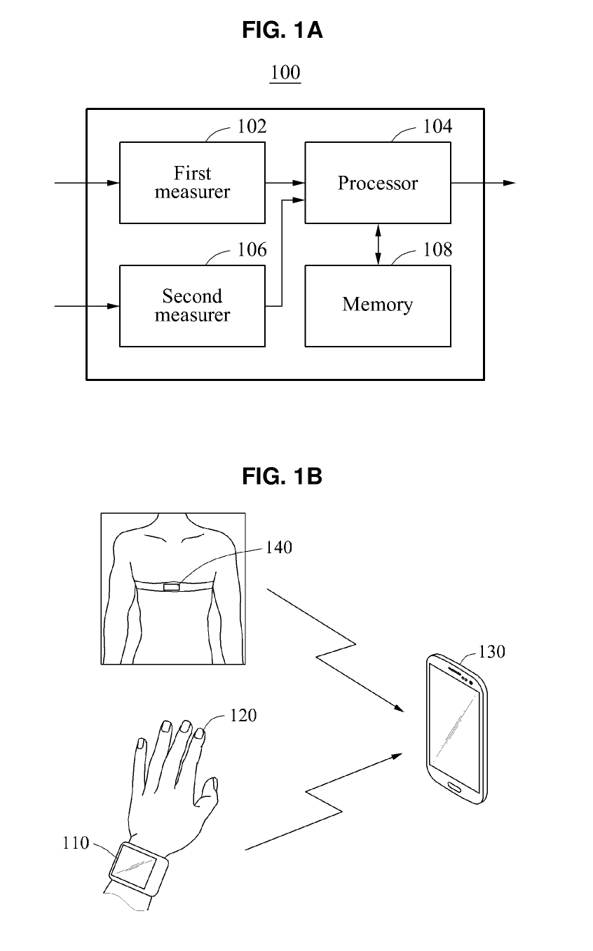

FIG. 1A is a block diagram illustrating an example of an apparatus 100 for estimating a heart rate. FIG. 1B illustrates examples of wearable devices 110 and 140 into which the apparatus 100 may be incorporated, and a mobile device 130 that may communicate with the apparatuses 110 and 140.

Referring to FIG. 1A, the apparatus 100 includes a first measurer 102, a processor 104, and a memory 108. The apparatus 100 further includes a second measurer 106. The first measurer 102, the processor 104, the second measurer 106, and the memory 108 are configured to communicate with one another through a bus or respective hardware communication elements.

The first measurer 102 measures movement information based on a physical activity of a user. The movement information includes, for example, a moving speed and a moving distance.

The first measurer 102 may include, for example, one or more of a global positioning system (GPS) sensor configured to sense a moving distance and a position of a user, an acceleration sensor, a pedal rotation sensor configured to measure a rotation speed, or a sensor configured to measure a stride length and a cadence. The first measurer 102 measures the movement information using one or more of the aforementioned sensors.

The second measurer 106 measures a heart rate (HR) signal of a user sensed based on the physical activity of the user. The first measurer 102 includes, for example, an electrocardiogram (ECG) sensor and a photoplethysmogram (PPG) sensor, or may be in communication with the same.

The physical activity of the user includes a general physical activity, for example, a stroll or shopping, and a graded load exercise, for example, running, jogging, or cycling.

The processor 104 estimates a heart rate of the user by applying the movement information measured by the first measurer 102 to a regression model set in advance. The regression model is based on a correlation between previous heart rate information and such movement information.

When the heart rate is unmeasured, or corresponding heart rate information is missing or lacking in the measured signal, or the heart rate is incorrectly measured by the second measurer 106, the processor 104 compensates for a portion of the measured heart rate corresponding to a heart-rate section (e.g., a measurement interval) in which the user's heart rate is unmeasured or incorrectly measured by applying or substituting the corresponding portion of the estimated heart rate for that heart rate section, so the compensated heart rate includes portions of the measured heart rate and one or more portions of the estimated heart rate.

The processor 104 evaluates a signal quality of the heart rate measured by the second measurer 106 based on at least one of a difference between the measured heart rate and the estimated heart rate and a ratio between the measured heart rate and the estimated heart rate. When the signal quality of the heart rate measured by the second measurer 106 is lower than a preset reference, the processor 104 substitutes the measured heart rate with the estimated heart rate.

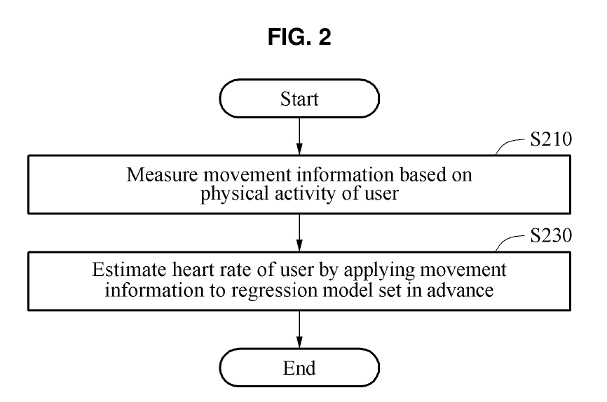

Also, the processor 104 performs one or more methods described with reference to FIGS. 2 through 10. The processor 104 may execute a program stored on a non-transitory medium or other memory and control the apparatus 100 to implement the one or more methods of FIGS. 2-10. For example, in an embodiment, a code of the program to be executed by the processor 104 is stored in the memory 108. The apparatus 100 may be connected to an external device, for example, a personal computer and a network, through an input and output hardware element of the apparatus 100, thereby performing a data exchange.

The memory 108 stores, for example, the movement information measured by the first measurer 102, the heart rate measured by the second measurer 106, and the heart rate estimated by the processor 104. The memory 108 may be a volatile memory and also be a non-volatile memory.

At least one of the methods described with reference to FIGS. 1 through 10 is implemented to be in a form of an application executed in a processor of a tablet computer, a smartphone, or a wearable device, or implemented to be in a form of a chip embedded in the smartphone or the wearable device.

Referring to FIG. 1B, the apparatus 100 may be included or embedded in a wearable device, such as wearable devices 110 and 140. In the example of FIG. 1B, the wearable device 110 is illustrated as a watch, and the wearable device 140 is illustrated as a chest strap. However, the apparatus 100 may be provided in other types of wearable devices. According to various examples, the wearable devices 110 and 140 may be a wrist-worn device provided in a form of a watch, a bracelet, or the like. Also, the wearable devices 110 and 140 may be provided in a form of a necklace, a chest-worn form, an ear-worn form, or any other forms. For example, when a user 120 wears the wearable devices 110 or 140 and performs an exercise, the apparatus 100 measures a heart rate at a wrist or a chest, respectively, of the user 120. In this example, when an unmeasured heart rate section or the mis-measured, i.e., incorrect, heart-rate section is included in the measured heart rate, the apparatus 100 compensates for the heart rate with regard to the unmeasured section or the mis-measured section based on the heart rate estimated by the processor 104.

The wearable device 110 or 140 including the apparatus 100 is interconnected with the mobile device 130 to share data with the mobile device 130. As an example, the measured heart rate of the user 120 or the heart rate estimated by the apparatus 100 is transferred to the mobile device 130.

In another example, the processor 104 is included in the mobile device 103, and a first measurer 102 and a second measurer 106 are included in each of the wearable devices 110 and 140. Alternatively, the first measurer 102 and the processor 104 of the apparatus 100 are included in the mobile device 130, and a second measurer 106 is included in each of the wearable devices 110 and 140.

In still another example, the apparatus 100 is entirely included in and operates in the mobile device 130 irrespective of a presence of the wearable devices 110 and 140.

The wearable devices 110 and 140 are configured to be worn on a body part, for example, a wrist, a chest, an ear, a neck or a finger, of the user 120 and measure the heart rate of the user 120 at or near that body part. The wearable device 110 or 140 amplifies and filters the measured heart rate. The wearable device 110 and 140 transmits the measured heart rate to the mobile device 130. When a signal quality of the measured heart rate is lower than a preset reference, the apparatus 100 substitutes the measured heart rate with the estimated heart rate or analyzes physical activity information of the user 120, based on the heart rate received from the wearable device 110 or 140.

The wearable devices 110 and 140 may each be connected with the mobile device 130 through a wireless link. The mobile device 130 and the wearable devices 110 and 140 may each include one or more wireless Internet interfaces, such as a wireless local area network (WLAN) interface, a Wi-Fi interface, a digital living network alliance (DLNA interface), a wireless broadband (WiBro) interface, a world interoperability for microwave access (WiMAX) interface, or a high-speed downlink packet access (HSDPA) interface, for example, and one or more short-range communication interfaces, such as a Bluetooth interface, a radio frequency identification (RFID) interface, an infrared data association (IrDA) interface, an ultra wideband (UWB) interface, a ZigBee interface, or a near field communication (NFC) interface, for example.

The mobile device 130 may be implemented as a tablet computer, a smart phone, or a personal digital assistant (PDA), for example. The mobile device 130 may be network equipment such as a server. The mobile device 130 may be a single server computer or a system similar thereto, or at least one server bank or server cloud distributed at different geographical locations.

The mobile device 130 receives various types of biosignals as well as a heart rate through the wearable devices 110 and 140 or any other measuring device.

FIG. 2 is a flowchart illustrating an example of a method of estimating a heart rate. Referring to FIG. 2, in operation S210, an apparatus for estimating a heart rate measures movement information based on a physical activity of a user. Hereinafter, any of the apparatuses for estimating a heart rate is also be respectively referred to as, for example, an “estimation apparatus.” The estimation apparatus measures the movement information using, for example, one or more of a GPS sensor, an acceleration sensor, a pedal rotation sensor, and a sensor for measuring a stride length and a cadence. The movement information includes, for example, a moving speed and a moving distance. The physical activity of the user includes a general physical activity, for example, a stroll or shopping, and a graded load exercise, for example, a daily activity in which a load of exercise increases.

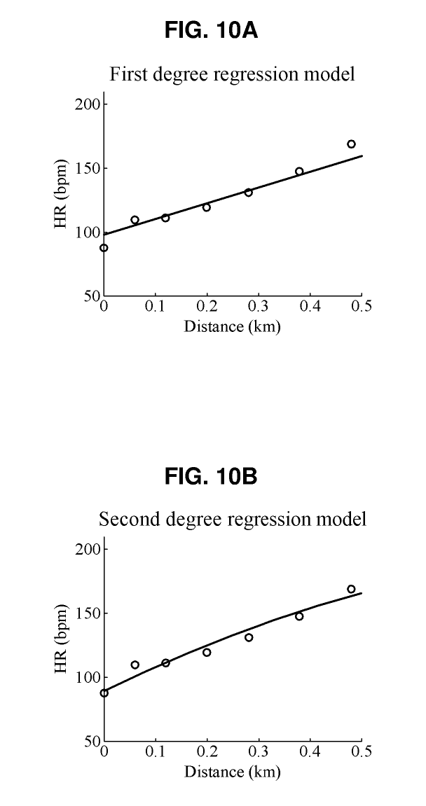

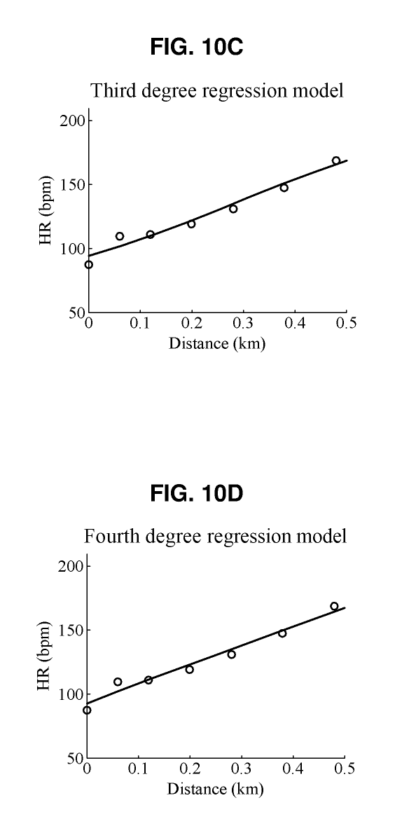

In operation S230, the estimation apparatus estimates a heart rate of the user by applying the movement information to a regression model set in advance. The estimation apparatus estimates the heart rate of the user by applying the movement information to a regression model of which a variable is the movement information such as the moving speed or the moving distance. The regression model is based on a correlation between a heart rate and movement information. Also, the regression model is one of a first degree regression model, a second degree regression model, and a third degree regression model, and a fourth degree regression model. An example of a correlation between a heart rate and movement information will be described with reference to graphs of FIG. 10.

The regression model may be a linear regression model or a non-linear regression model. The regression model includes, for example, a first regression model set based on a correlation between an average heart rate and average moving information of a plurality of users and a second regression model set based on a correlation between the heart rate and the movement information of the user. The second regression model is, for example, a regression model obtained by performing a personalized correction on the first regression model. Either of such regression models may be based, at least partially on previous heart rate and/or physical movement measurements or categories.

The regression model is provided in a form of, for example, Y=α×X+β. In this example, a value to be applied to X is the movement information and Y is the estimated heart rate. In the regression model, coefficients (α, β) are determined based on a degree of the regression model.

The estimation apparatus estimates the heart rate of the user by applying the movement information to one of the first regression model and the second regression model based on whether the personalized correction is to be performed.

In an example, the estimation apparatus receives body information of the user and applies the movement information and the body information to the regression model, thereby estimating the heart rate. In this example, the body information may be directly input by the user, e.g., through the user interface of any of the devices 110, 130 and 140 of FIG. 1B. Alternatively, the body information may be one or more prestored values. The body information may be updated. The body information may include, for example, a gender, an age, a height, a weight, and a body mass index (BMI) of the user. The BMI may be obtained by dividing a weight by a square of a height. In this example, a unit of the weight is a kilogram (kg) and a unit of the square of the height is a square meter (m²).

In a case in which the movement information and the body information of the user are applied to the regression model, the regression model may be, for example, Y=α1 ×X1+α2×X2+β. In this example, a value to be applied to X1 is the movement information, a value to be applied to X2 is the body information of the user, and Y is the estimated heart rate. In the regression model, coefficients (α1, α2, β) are determined based on a type of regression model, for example, the first degree regression model, the second degree regression model, a linear regression model, a the non-linear regression model.

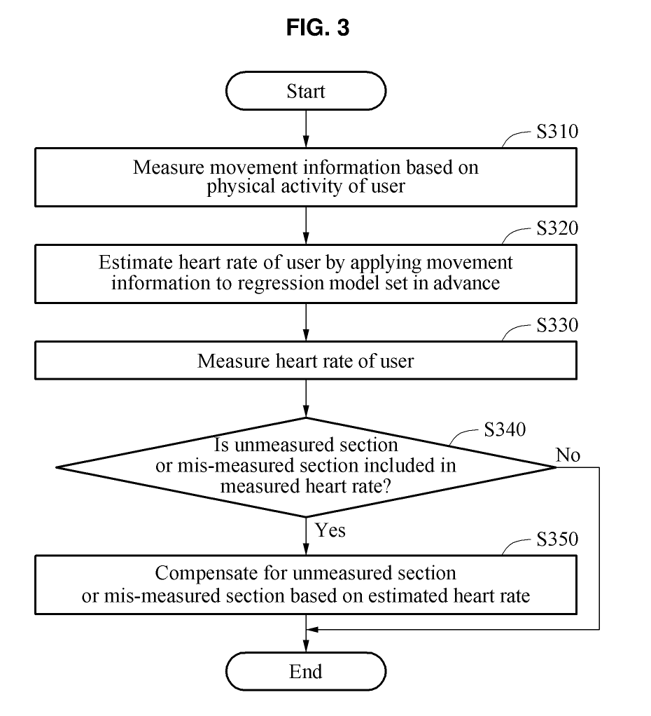

FIG. 3 is a flowchart illustrating another example of a method of estimating a heart rate. Since the descriptions provided with reference to operations S210 and S230 of FIG. 2 are also applicable here, repeated descriptions with respect to corresponding operations S310 and S320 of FIG. 3 will be omitted.

In operation S330, an estimation apparatus measures a heart rate of the user. The estimation apparatus is, for example, a wearable device including a heart rate sensing device or a heart rate system provided in a form such as a watch-type form, a bracelet-type form, a chest-type form, an in-ear type form, or the like, or a mobile device connected with the wearable device through a wired or wireless communication link. The heart rate sensing device may include, for example, a PPG sensor and an ECG sensor. The estimation apparatus measures the heart rate of the user using various types of heart rate sensing devices or heart rate systems.

The measured heart rate may include noise occurring due to a movement of a sensor or motion noise. The estimation apparatus detects heart rate components in lieu of such noise based on, for example, a maximum peak picking method in which a window size including one heart rate cycle is determined and a maximum peak point is detected from an ECG or a pulse wave signal of a corresponding heart rate section, knowledge-based rules in which features of the ECG or the pulse wave signal are applied through an intellectualization, an adaptive threshold method, and a template matching method in which a user standard waveform, for example, a template, is determined, and a detection is performed based on a degree of correlation.

In operation S340, the estimation apparatus determines whether an unmeasured section or a mis-measured heart-rate section is included in the measured heart rate, e.g., in a corresponding heart rate biosignal. For example, the estimation apparatus selects models indicating an ECG or a pulse wave and trains learning models based on previous heart rate data or previous and current heart rate data. The estimation apparatus determines whether a heart rate measured using the learning model includes a heart rate section in which the heart rate is unmeasured or a heart rate section in which the heart rate is incorrectly measured due to the noise.

As a result of the determining of operation S340, in response to the measured heart rate being determined to not include such an unmeasured section or mis-measured section, the estimation apparatus terminates its overall operation.

As a result of the determining of operation S340, in response to the measured heart rate being determined to include such an unmeasured or mis-measured heart rate section, in operation S350, the estimation apparatus compensates for a portion of the heart rate corresponding to the unmeasured heart rate section or the mis-measured heart rate section by applying a corresponding portion of the estimated heart rate obtained from operation S320 or substituting a corresponding portion of the heart rate from operation S320 for that heart rate section. In an example, an acceleration sensor and a GPS sensor included in a mobile terminal may be used to readily compensate for a value of an heart rate unmeasured section or a heart rate mis-measured section caused by the motion noise and the movement of the sensor during an exercise without need for separate device or assistance.

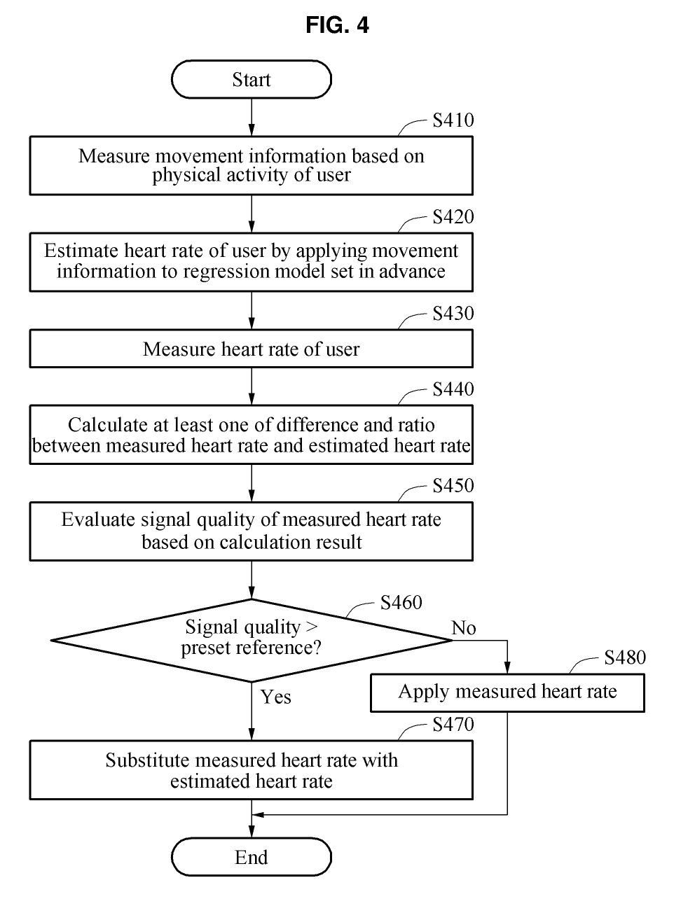

FIG. 4 is a flowchart illustrating an example of a method of estimating a heart rate. Since the descriptions provided with reference to operations S310 through S330 of FIG. 3 are also applicable here, repeated descriptions with respect to corresponding operations S410 through S430 of FIG. 4 will be omitted.

In operation S440, an estimation apparatus calculates at least one of a difference between a heart rate measured in operation S430 and a heart rate estimated in operation S420 (“heart rate difference”), and a ratio between the measured heart rate and the estimated heart rate (“heart rate ratio”).

In operation S450, the estimation apparatus evaluates a signal quality of the measured heart rate based on a calculation result of operation S440. Also, the estimation apparatus evaluates a signal quality of the measured heart rate based on, for example, a method of detecting a heart rate from an ECG signal measured by one lead wire based on a plurality of heart rate detection algorithms and comparing detection results, a method of detecting a heart rate from an ECG signal measured by a plurality of lead wires based on a plurality of heart rate detection algorithms and comparing detection results, a method of detecting a heart rate based on a kurtosis of a heart rate signal included in a preset window, or a method of detecting a heart rate based on a spectrum distribution of a heart rate signal.

The estimation apparatus detects at least one of an invalid heart rate section and a valid heart rate section from the measured heart rate based on at least one of the calculated heart rate difference and the calculated heart rate ratio. For example, the estimation apparatus compares the calculated heart rate difference or the calculated heart rate ratio to respective preset reference thresholds. As a result of the comparing, the estimation apparatus determines a heart rate section in which the difference or the ratio has a value greater than the preset reference to be the invalid heart rate section.

In operation S460, the estimation apparatus compares the signal quality of the measured heart rate to a preset reference. In a comparison result of operation S460, in response to the signal quality being lower than the preset reference, the estimation apparatus substitutes the measured heart rate with the estimated heart rate in operation S470.

In the comparison result of operation 460, in response to the signal quality being higher than or equal to the preset reference, the estimation apparatus applies the measured heart rate in operation S480.

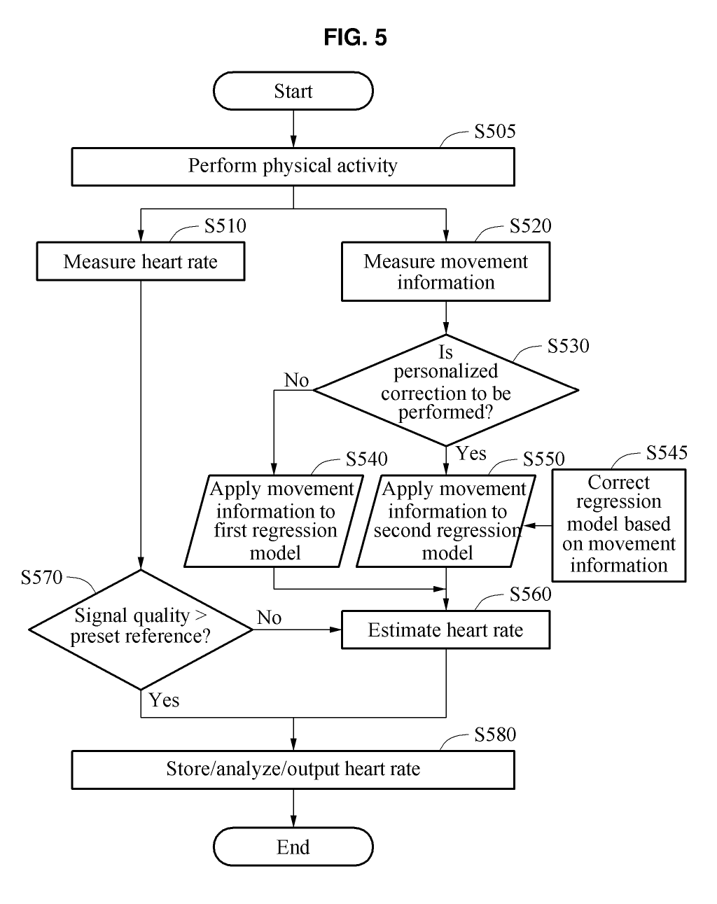

FIG. 5 illustrates an example of estimating, storing, analyzing, and outputting a heart rate. Referring to FIG. 5, when a user wearing an estimation apparatus performs various physical activities, for example, strolling, jogging, running, and cycling, in operation S505, the estimation apparatus measures a heart rate of the user in operation S510 and simultaneously measures movement information in operation S520. Based on the movement information including, for example, a moving speed and a moving distance measured with a relatively high accuracy during an exercise, the estimation apparatus robustly estimates the heart rate against motion noise and a movement of a sensor.

In operation S530, the estimation apparatus determines whether a personalized correction is to be performed on a regression model. The estimation apparatus determines whether a first regression model or a second regression model is/will be used to estimate the heart rate based on whether the personalized correction is to be performed. When the personalized correction is not to be performed on the regression model, the estimation apparatus applies movement information to the first regression model in operation S540. When the personalized correction is to be performed on the regression model, the estimation apparatus applies the movement information to the second regression model in operation S550. In this example, the second regression model is the regression model corrected to be personalized for the user based on the measured heart rate and/or the movement information of the user in operation S545.

In operation S560, the estimation apparatus estimates the heart rate based on a result of applying the movement information to each regression model, for example, the first regression model or the second regression model. In operation S580, the estimation apparatus stores, analyzes, or outputs the estimated heart rate.

In operation S570, the estimation apparatus a signal quality of the heart rate measured in operation 510 to a preset reference. In the comparison result of operation 570, in response to the signal quality being determined to be higher than the preset reference, in operation S580, the estimation apparatus stores, analyzes, or outputs the heart rate measured in operation S510.

In a comparison result of operation S570, in response to the signal quality being determined to be lower than or equal to the preset reference, in operation S580, the estimation apparatus stores, analyzes, or outputs the heart rate estimated in operation S560.

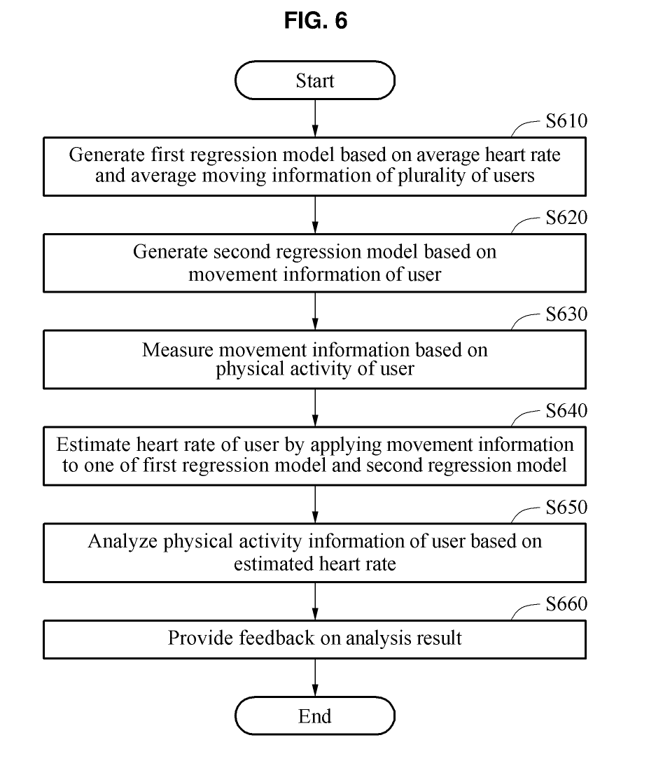

FIG. 6 illustrates an example of a method of estimating a heart rate. Referring to FIG. 6, in operation S610, an estimation apparatus generates a first regression model based on an average heart rate and average moving information of a plurality or sampling of users. The first regression model is, for example, a regression model previously learned based on a correlation between the average heart rate and the average moving information of the plurality of users.

In operation S620, the estimation apparatus generates a second regression model personalized a user based on movement information of the user. By correcting the first regression model based on the movement information of the user, the estimation apparatus generates the second regression model. The estimation apparatus calculates a regression equation using a regression model having, for example, the movement information of the user as an independent variable. The estimation apparatus generates the second regression model by correcting coefficients of the regression equation calculated based on the movement information, the measured heart rate, and/or body information of the user.

In operation S630, the estimation apparatus measures the movement information based on a physical activity of the user. In an example, the estimation apparatus corrects the second regression model generated in operation S620 based on a measured heart rate and currently measured movement information of the user.

In operation S640, the estimation apparatus estimates the heart rate of the user by applying the movement information to one of the first regression model and the second regression model. The estimation apparatus estimates the heart rate of the user by applying the movement information to one of the first regression model and the second regression model based on whether a personalized correction is to be performed.

In operation S650, the estimation apparatus analyzes physical activity information of the user based on the estimated heart rate. As an example, in response to the estimated heart rate being lower than a preset reference, the estimation apparatus determines that a physical activity or exercise ability of the user is relatively low.

In operation S660, the estimation apparatus provides a feedback on an analysis result of operation S650 to the user, e.g., through the user interface of the estimation apparatus. For example, in an embodiment, the estimation apparatus may further select an exercise program suitable for the analyzed physical activity from prestored exercise programs and provides a feedback on the selected exercise program to the user.

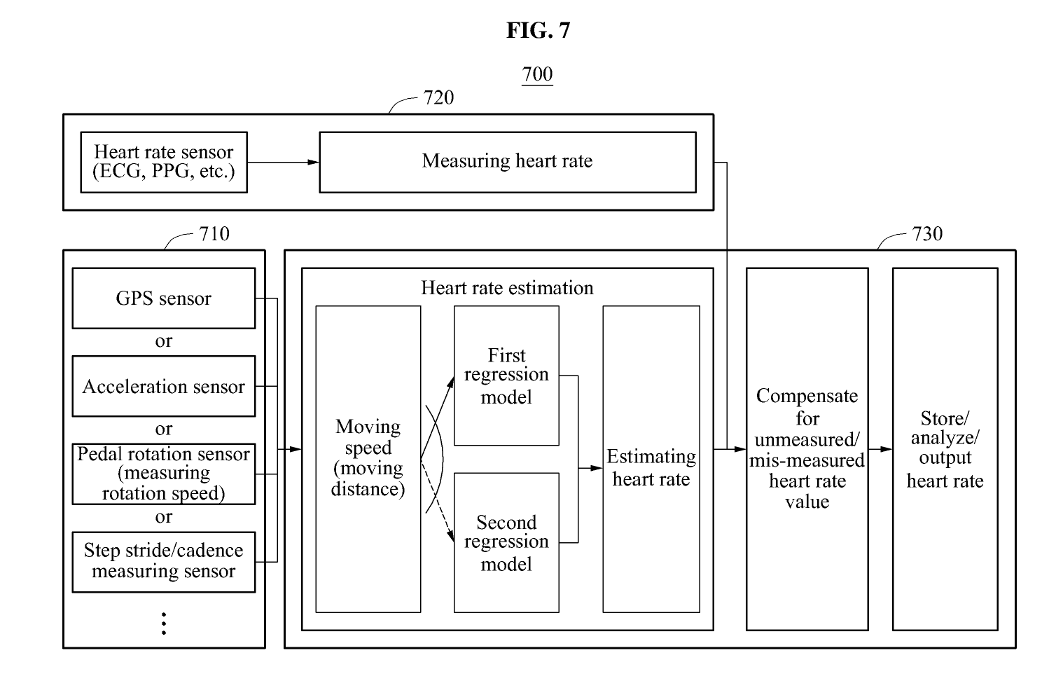

FIG. 7 illustrates an example of operating an apparatus 700 for estimating a heart rate. Referring to FIG. 7, the apparatus compensates for a heart rate unmeasured or mis-measured due to motion noise and a movement of a sensor which may occur during various physical activities. The apparatus 700 includes a first measurer 710, a second measurer 720, and a processor 730.

The apparatus 700 uses the first measurer 710 to measure movement information including, for example, a moving speed and a moving distance of a user with a reduced error in measurement and an increased reliability. Also, the apparatus 700 measures a heart rate of the user by using the second measurer 720 including a heart rate sensor.

The processor 730 estimates the heart rate using a regression model based on whether a personalized correction is to be performed. For example, the processor 730 uses a first regression model that is a common regression model when the personalized correction is not performed, and uses a personalized second regression model that is a personalized regression model when the personalized correction of the common regression models is performed, for example.

In response to a heart rate corresponding to a heart rate unmeasured section or a heart rate mis-measured section being determined to be included in the heart rate measured by the first measurer 710, the processor 730 compensates for the heart rate with regard to the heart rate unmeasured section or the heart rate mis-measured section based on the estimated heart rate, or substitutes the estimated heart rate for the heart rate unmeasured section or the heart rate mis-measured section in the measured heart rate. The processor 730 stores, analyzes, or outputs the estimated heart rate.

In an example, by compensating for the unmeasured heart rate section or the mis-measured heart rate section, the apparatus 700 acquires and analyzes the heart rate during various physical activities with a reduced error in measurement and an increased reliability.

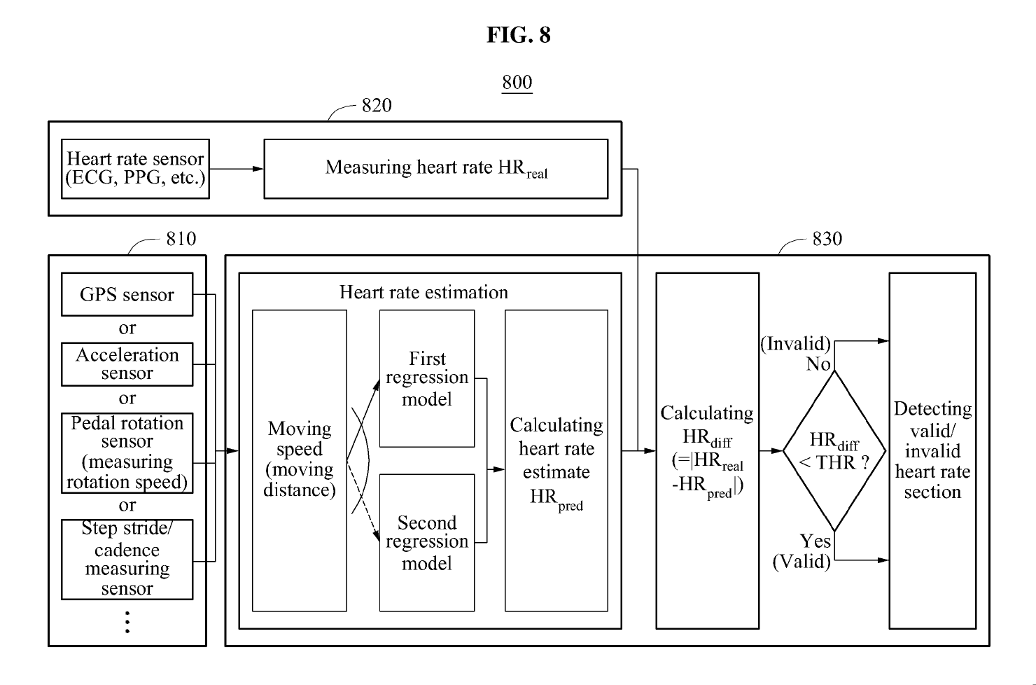

FIG. 8 illustrates example of operations of an apparatus 800 for estimating a heart rate. Referring to FIG. 8, the apparatus 800 evaluates a signal quality of a heart rate based on movement information of a user. The apparatus 800 includes a first measurer 810, a second measurer 820, and a processor 830. The above disclosure regarding FIGS. 1-7 is applicable to the apparatus 800, though embodiments are not limited thereto.

In response to movement information of a user being measured by the first measurer 810, the processor 830 calculates a heart rate estimate HR_{pred} using a regression model based on whether a personalized correction is to be performed.

The second measurer 820 measures an actual heart rate HRᵣₑₐₗ.

The processor 830 calculates a difference HR_{diff} between the heart rate HRᵣₑₐₗ and the heart rate estimate HR_{pred} and determines whether the difference HR_{diff} is less than or equal to a threshold THR set in advance. In response to the difference HR_{diff} being less than or equal to the threshold THR, the processor 830 determines that the heart rate HRᵣₑₐₗ is measured accurately. In response to the difference HR_{diff} being greater than the threshold THR, the processor 830 determines that the heart rate HRᵣₑₐₗ is measured inaccurately. The processor 830 detects a heart rate section corresponding to the difference HR_{diff} less than or equal to the threshold THR to be a valid heart rate section, and detects a section corresponding to the difference HR_{diff} greater than the threshold THR to be an invalid heart rate section.

The processor 830 also detects the valid heart rate section and the invalid heart rate section by calculating a ratio between the heart rate HRᵣₑₐₗ and the heart rate estimate HR_{pred} in lieu of calculating the difference HR_{diff} between the heart rate HRreal and the heart rate estimate HR_{pred}.

In an example, the processor 830 performs a heart rate correction on the invalid heart rate section. For example, the processor 830 performs the heart rate correction based on well-known signal compensation algorithms. In another example, the processor 830 substitutes the heart rate in the invalid heart rate section with the estimated heart rate for this section based on the movement information of the user.

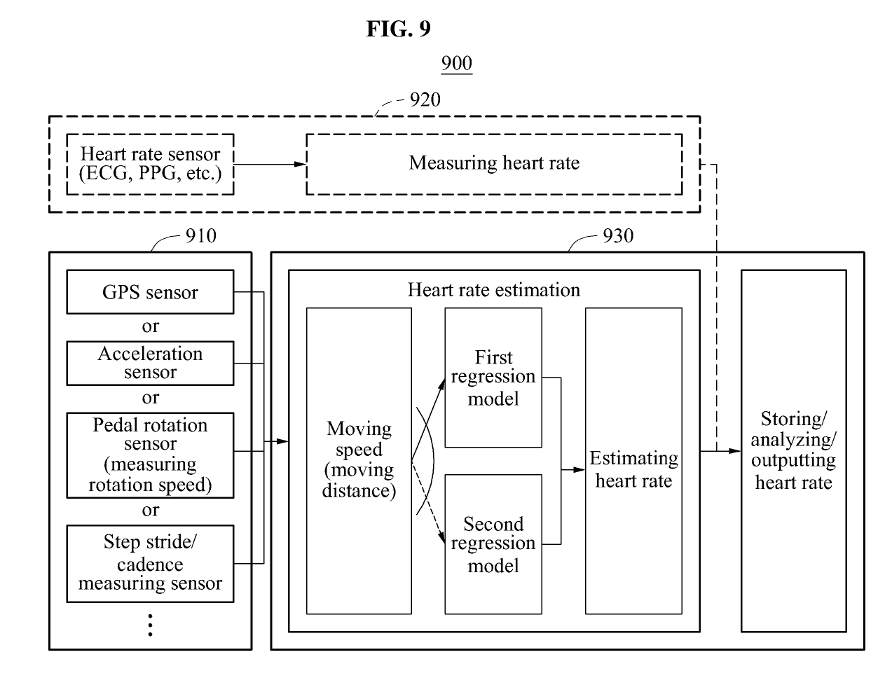

FIG. 9 illustrates still another example of operations of an apparatus 900 for estimating a heart rate. Referring to FIG. 9, the apparatus 900 substitutes an estimated heart rate with an actual heart rate when a user performs a physical activity in which measuring a heart rate measurement is difficult. The apparatus 900 includes a first measurer 910, a second measurer 920, and a processor 930.

When a user performs a physical activity in which measuring a heart rate measurement is difficult, for example, a horse-riding and a swimming, the second measurer 920 is practically capable of measuring the heart rate of the user even though the first measurer 910 is capable of measuring the movement information of the user. In this example, the processor 930 estimates the heart rate of the user by applying the movement information to a preset regression model. The processor 930 substitutes an actual heart rate of the user with the estimated heart rate.

FIGS. 10A-10D illustrate graphs representing examples of a correlation between a moving distance and an actually measured heart rate using regression models through a personalization operation. In each of the respective graphs of FIGS. 10A-10D, an X axis represents a moving distance and a Y axis represents a measured heart rate.

In an example, an estimation apparatus acquires regression models based on a treadmill moving speed or a treadmill moving distance and a heart rate measured during a 012052.1359 heart-lung strength assessment, for example, a two-minute unit graded exercise protocol. The estimation apparatus derives or acquires, e.g., from the memory of the apparatus 900, a first degree regression model (FIG. 10A), a second degree regression model (FIG. 10B), a third degree regression model (FIG. 100), and a fourth degree regression model (FIG. 10D) based on, for example, a one-minute unit average heart rate and a moving distance with respect to all user data included in a training database (DB). In this example, in each regression model, a dependent variable is a measured heart rate, and an independent variable is movement information including, for example, the moving distance or a moving speed.

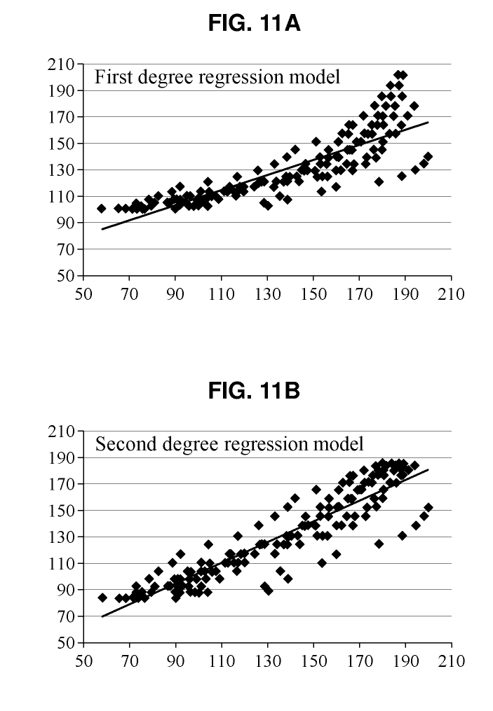

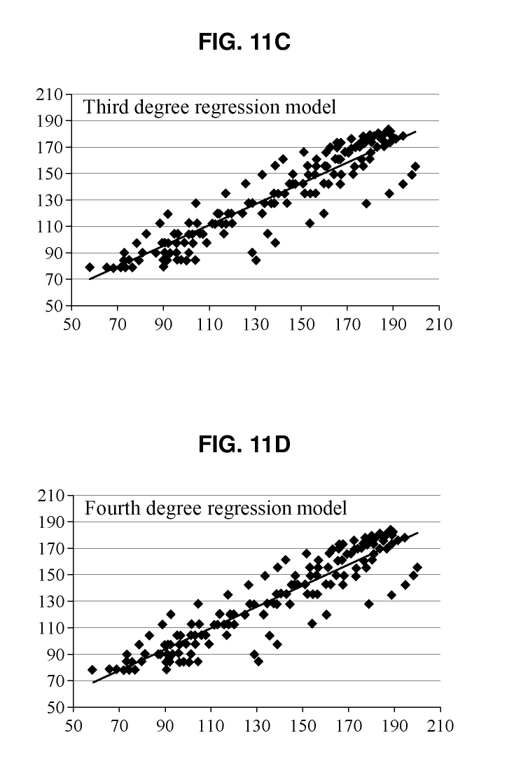

The estimation apparatus calculates a heart rate estimated based on a result value of each regression model. The estimation apparatus analyze an error rate and a correlation between an actual heart rate and the heart rate estimated based on each regression model. The correlation between the actual heart rate and the heart rate estimated based on each regression model will be described with reference to FIGS. 11A-11D.

FIGS. 11A-11D illustrate graphs representing examples of a correlation between an actual heart rate and an estimated heart rate using respective first through fourth regression models through a generalization operation. In each of the respective graphs of FIGS. 11A-11D, an X axis represents an actual heart rate and a Y axis represents a heart rate estimated using a regression model.

Referring to FIGS. 11A-11D, a tendency of an actual heart rate is similar to a tendency of a heart rate estimated using a regression model. Also, a correlation between the actual heart rate and the estimated heart rate increases according to an increase in a degree of the regression model. Thus, it can be known from the graphs that a fourth degree regression model (FIG. 11D) provides a higher performance when compared to a first degree regression model (FIG. 11A).

The apparatuses, units, modules, devices, and other components illustrated in FIGS. 1A, 1B and 7-9 (e.g., the first measurers 102, 710, 810 and 910, the processors 104, 730, 830, 930, the second measurers 106, 720, 820 and 920, and the memory 108) that perform the operations described herein with respect to FIGS. 2-11D are implemented by hardware components. Examples of hardware components include controllers, sensors, generators, drivers, memories, comparators, arithmetic logic units, adders, subtractors, multipliers, dividers, integrators, and any other electronic components known to one of ordinary skill in the art. In one example, the hardware components are implemented by computing hardware, for example, by one or more processors or computers. A processor or computer is implemented by one or more processing elements, such as an array of logic gates, a controller and an arithmetic logic unit, a digital signal processor, a microcomputer, a programmable logic controller, a field-programmable gate array, a programmable logic array, a microprocessor, or any other device or combination of devices known to one of ordinary skill in the art that is capable of responding to and executing instructions in a defined manner to achieve a desired result. In one example, a processor or computer includes, or is connected to, one or more memories storing instructions or software that are executed by the processor or computer. Hardware components implemented by a processor or computer execute instructions or software, such as an operating system (OS) and one or more software applications that run on the OS, to perform the operations described herein with respect to FIGS. 2-11 D. The hardware components also access, manipulate, process, create, and store data in response to execution of the instructions or software. For simplicity, the singular term “processor” or “computer” may be used in the description of the examples described herein, but in other examples multiple processors or computers are used, or a processor or computer includes multiple processing elements, or multiple types of processing elements, or both. In one example, a hardware component includes multiple processors, and in another example, a hardware component includes a processor and a controller. A hardware component has any one or more of different processing configurations, examples of which include a single processor, independent processors, parallel processors, single-instruction single-data (SISD) multiprocessing, single-instruction multiple-data (SIMD) multiprocessing, multiple-instruction single-data (MISD) multiprocessing, and multiple-instruction multiple-data (MIMD) multiprocessing.

The methods illustrated in FIGS. 2-11D that perform the operations described herein with respect to FIGS. 1A, 1B and 7-9 are performed by computing hardware, for example, by one or more processors or computers, as described above executing instructions or software to perform the operations described herein.

Instructions or software to control a processor or computer to implement the hardware components and perform the methods as described above are written as computer programs, code segments, instructions or any combination thereof, for individually or collectively instructing or configuring the processor or computer to operate as a machine or special-purpose computer to perform the operations performed by the hardware components and the methods as described above. In one example, the instructions or software include machine code that is directly executed by the processor or computer, such as machine code produced by a compiler. In another example, the instructions or software include higher-level code that is executed by the processor or computer using an interpreter. Programmers of ordinary skill in the art can readily write the instructions or software based on the block diagrams and the flow charts illustrated in the drawings and the corresponding descriptions in the specification, which disclose algorithms for performing the operations performed by the hardware components and the methods as described above.

The instructions or software to control a processor or computer to implement the hardware components and perform the methods as described above, and any associated data, data files, and data structures, are recorded, stored, or fixed in or on one or more non-transitory computer-readable storage media. Examples of a non-transitory computer-readable storage medium include read-only memory (ROM), random-access memory (RAM), flash memory, CD-ROMs, CD-Rs, CD+Rs, CD-RWs, CD+RWs, DVD-ROMs, DVD-Rs, DVD+Rs, DVD-RWs, DVD+RWs, DVD-RAMs, BD-ROMs, BD-Rs, BD-R LTHs, BD-REs, magnetic tapes, floppy disks, magneto-optical data storage devices, optical data storage devices, hard disks, solid-state disks, and any device known to one of ordinary skill in the art that is capable of storing the instructions or software and any associated data, data files, and data structures in a non-transitory manner and providing the instructions or software and any associated data, data files, and data structures to a processor or computer so that the processor or computer can execute the instructions. In one example, the instructions or software and any associated data, data files, and data structures are distributed over network-coupled computer systems so that the instructions and software and any associated data, data files, and data structures are stored, accessed, and executed in a distributed fashion by the processor or computer.

While this disclosure includes specific examples, it will be apparent to one of ordinary skill in the art that various changes in form and details may be made in these examples without departing from the spirit and scope of the claims and their equivalents. The examples described herein are to be considered in a descriptive sense only, and not for purposes of limitation. Descriptions of features or aspects in each example are to be considered as being applicable to similar features or aspects in other examples. Suitable results may be achieved if the described techniques are performed in a different order, and/or if components in a described system, architecture, device, or circuit are combined in a different manner, and/or replaced or supplemented by other components or their equivalents. Therefore, the scope of the disclosure is defined not by the detailed description, but by the claims and their equivalents, and all variations within the scope of the claims and their equivalents are to be construed as being included in the disclosure.

What is claimed is:

1. A method of estimating a heart rate, the method comprising:
measuring, using a processor, movement information based on a physical activity of a user; and
estimating, using the processor, a heart rate of the user by applying the movement information to a preset regression model, wherein the regression model is based on a correlation between heart rates and movement information.

2. The method of claim 1, wherein the movement information comprises at least one of a measured moving speed or a moving distance for the user.

3. The method of claim 1, wherein the regression model comprises at least one of a first regression model preset based on a correlation between average heart rates and average moving information of a plurality of users and a second regression model preset based on a correlation between heart rates and at least previous movement information of the user.

4. The method of claim 3, wherein the estimating comprises estimating the heart rate of the user by applying the movement information to one of the first regression model and the second regression model based on whether a personalized correction is determined to be performed.

5. The method of claim 1, further comprising:
generating the regression model so as to be personalized for the user based on previous movement information of the user.

6. The method of claim 5, wherein the generating comprises generating a second regression model by correcting a first regression model based on the previous movement information of the user.

7. The method of claim 1, further comprising:
measuring the heart rate of the user.

8. The method of claim 7, further comprising:
compensating for a portion of the heart rate of the user determined to be an unmeasured or mis-measured heart rate section based on the estimated heart rate.

9. The method of claim 7, further comprising:
evaluating a signal quality of the measured heart rate based on at least one of a difference between the measured heart rate and the estimated heart rate, or a ratio between the measured heart rate and the estimated heart rate.

10. The method of claim 9, further comprising:
substituting the portion of the measured heart rate with a corresponding portion of the estimated heart rate in response to the signal quality being lower than a preset reference.

11. The method of claim 9, wherein the evaluating of the signal quality further comprises detecting at least one of an invalid heart rate section or a valid heart rate section from the measured heart rate, based on at least one of the difference and the ratio.

12. The method of claim 11, wherein the detecting comprises:
comparing the difference or the ratio to a preset reference; and
determining that a heart rate section of the measured heart rate in which the difference or the ratio has a value greater than the preset reference is the invalid heart rate section, based on a result of the comparing.

13. The method of claim 1, further comprising:
analyzing physical activity information of the user based on the estimated heart rate; and
providing a feedback on a result of the analyzing.

14. The method of claim 1, further comprising:
receiving body information of the user,
wherein the estimating comprises estimating the heart rate of the user by applying the movement information and the body information of the user to the regression model.

15. The method of claim 14, wherein the body information comprises at least one of a gender, an age, a height, a weight, a body mass index (BMI), or a stable-state heart rate.

16. A non-transitory computer readable medium, comprising programmed instructions configured to control one or more processor devices to implement the method of claim 1.

17. An apparatus for estimating a heart rate, the apparatus comprising:
a first measurer configured to measure movement information of a user based on a physical activity of the user; and
a processor configured to estimate a heart rate of the user by applying the movement information to a preset regression model,
wherein the regression model is based on a correlation between heart rates and movement information.

18. The apparatus of claim 17, further comprising:
a second measurer configured to measure the heart rate of the user.

19. The apparatus of claim 18, wherein, the processor is configured to compensate for the heart rate of the user based on the estimated heart rate, in response to a portion of the heart rate of the user being determined to be unmeasured or measured incorrectly.

20. The apparatus of claim 18, wherein the processor is configured to:
evaluate a signal quality of the measured heart rate based on at least one of a difference between the measured heart rate and the estimated heart rate, or a ratio between the measured heart rate and the estimated heart rate; and
substitute the portion of measured heart rate with a corresponding portion of the estimated heart rate in response to the signal quality being lower than a preset reference.
